(19) United States
(12) Patent Application Publication
(10) Pub. No.: US 2017/0226458 A1
(43) Pub. Date: Aug. 10, 2017

(54) SPHEROID TRAP INSERT

(71) Applicants: Feng Li, Shrewsbury, MA (US); Allison Jean Tanner, Portsmouth, NH (US)

(72) Inventors: Feng Li, Shrewsbury, MA (US); Allison Jean Tanner, Portsmouth, NH (US)

(21) Appl. No.: 15/498,018

(22) Filed: Apr. 26, 2017

Related U.S. Application Data
(63) Continuation of application No. PCT/US15/58106, filed on Oct. 29, 2015.
(60) Provisional application No. 62/072,094, filed on Oct. 29, 2014.

(51) Int. Cl. C12M 1/12; C12M 1/32; C12M 1/00; C12N 5/00

(52) U.S. Cl. CPC C12M 25/04; C12N 5/0062; C12M 23/12; C12M 23/20; C12M 29/04

(57) ABSTRACT

A cell culture well insert may include a frame defining a first open end, a second open end, and at least one support extending therebetween. The cell culture well insert may also include a fluid permeable mesh coupled to the frame and disposed across the second open end. The mesh may define pores that have an average pore size in a range from 10 micrometers to 100 micrometers.

This is a continuation application of International Patent Application Serial No. PCT/US15/58106 filed on Oct. 29, 2015, which claims the benefit of priority to U.S. Provisional Application Ser. No. 62/072,094, filed on Oct. 29, 2014, the contents of which are relied upon and incorporated herein by reference in their entirety, and the benefit of priority under 35 U.S.C. §120 is hereby claimed.

### FIELD

The present disclosure relates to apparatuses, systems and methods for culturing cells.

### TECHNICAL BACKGROUND

Cells cultured in three dimensions, such as spheroids, can exhibit more in-vivo like functionality than their counterparts cultured in two dimensions as monolayers. In two dimensional cell culture systems, cells can attach to a substrate on which they are cultured. However, when cells are grown in three dimensions, such as spheroids, the cells interact with each other rather than attaching to the substrate. Accordingly, cell culture media exchanges for culture systems in which cells are cultured as spheroids that are mobile in suspension can present challenges.

### BRIEF SUMMARY

In accordance with various embodiments of the present disclosure, cell culture inserts configured to allow exchange of culture media in wells containing spheroids without aspirating or damaging the spheroids are described. The cell culture inserts can include a frame having a first open end, a second open end, and at least one support extending between the first open end and the second open end. The inserts also include a fluid permeable mesh coupled to the frame and disposed across the opening of the second end. The mesh defines pores having an average pore size in a range from 10 micrometers to 100 micrometers, e.g., 10, 20, 50 or 100 micrometers, including ranges between any of the foregoing values. In embodiments, the pores are sized to allow individual cells to pass, but to prevent spheroid cell clusters from passing.

Additional features and advantages of the subject matter of the present disclosure will be set forth in the detailed description which follows, and in part will be readily apparent to those skilled in the art from that description or recognized by practicing the subject matter of the present disclosure as described herein, including the detailed description which follows, the claims, as well as the appended drawings.

It is to be understood that both the foregoing general description and the following detailed description present embodiments of the subject matter of the present disclosure, and are intended to provide an overview or framework for understanding the nature and character of the subject matter of the present disclosure as it is claimed. The accompanying drawings are included to provide a further understanding of the subject matter of the present disclosure, and are incorporated into and constitute a part of this specification. The drawings illustrate various embodiments of the subject matter of the present disclosure and together with the description serve to explain the principles and operations of the subject matter of the present disclosure. Additionally, the drawings and descriptions are meant to be merely illustrative, and are not intended to limit the scope of the claims in any manner.

### BRIEF DESCRIPTION OF THE DRAWINGS

The following detailed description of specific embodiments of the present disclosure can be best understood when read in conjunction with the following drawings, where like structure is indicated with like reference numerals and in which:

FIG. 1A is a perspective view of an embodiment of a cell culture apparatus having wells.

FIG. 1B is a cross-sectional view of an embodiment of the apparatus depicted in FIG. 1A.

FIG. 1C is a schematic top view of cells grown in wells of an embodiment of a structured surface;

FIG. 2A is a perspective view of an embodiment of a sphere trap or well insert;

FIG. 2B and 2C are top views of embodiments of the well insert of FIG. 2A depicting two different shaped meshes;

FIG. 3 is a schematic cross-sectional view of an embodiment of a plurality of wells and a plurality of well inserts;

FIG. 4 is a schematic cross-sectional view of an embodiment of a well insert and a well including a plurality of sub-wells;

FIG. 5 is perspective view of an embodiment of a cell culture apparatus having a plurality of wells and a well insert; and

FIG. 6 is a schematic drawing illustrating a method for using an embodiment of an apparatus as described herein.

FIG. 7 is a side view of an array of wells containing spheroids, in an embodiment.

FIG. 8 is a schematic drawing illustrating an exemplary method for using an embodiment of an apparatus as described herein.

### DETAILED DESCRIPTION

Reference will now be made in greater detail to various embodiments of the subject matter of the present disclosure, some embodiments of which are illustrated in the accompanying drawings. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar to other numbered components.

The present disclosure describes, among other things, cell culture apparatuses having a substrate defining one or more wells in addition to a cell culture well insert that may be positioned within at least one of the one or more of wells.

In some embodiments, the wells may be configured such that cells cultured in the wells form spheroids. For example, the wells can be non-adherent to cells to cause the cells in the wells to associate with each other and form spheroid clusters. The spheroids may expand to size limits imposed by the geometry of the cells. In some embodiments, the wells may be coated with an ultra-low binding material to make the wells non-adherent to cells. Because the cells are non-adherent to the surface of the wells, exchange of cell culture media without aspirating or disturbing the spheroid can be difficult.

In some embodiments, the cell culture apparatuses with which an insert as described herein may be employed is a multi-well round bottom plate, such as a 96-well round bottom plate.

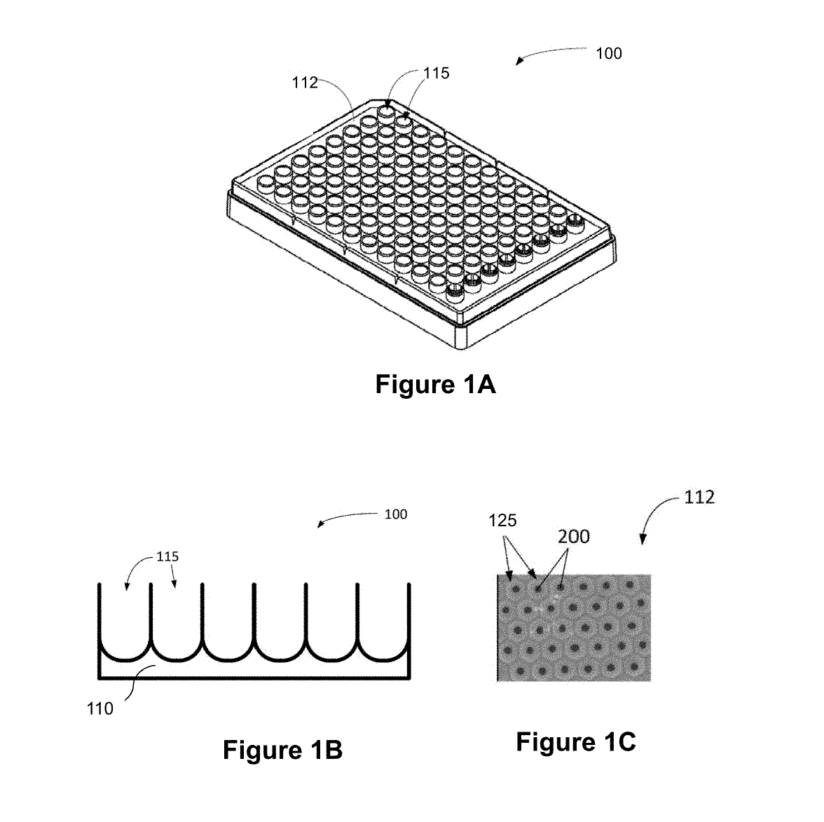

An embodiment of a cell culture apparatus 100 including a plurality of wells 115 is shown in FIGS. 1A and 1B. The plurality of wells 115 may have a variety of different arrangements. For example the plurality of wells 115 may define a pattern that is stacked, hexagonal close-packed, etc. Specifically, the structured surface 112 defining the wells 115 may include an array of stacked well structures as shown in FIG. 1A, showing the surface 112 forming wells 115. A side cross-sectional view of the cell culture apparatus 100 of FIG. 1A is shown in FIG. 1B. Each of the plurality of wells 115 drops below a major surface of the cell culture apparatus 100 and provides a position to form spheroids, as described above.

FIG. 1C is a schematic drawing showing cells 200 grown in wells 115 of an embodiment of a structured surface 112 including an array of hexagonal close-packed well structure. In some preferred embodiments, the cells 200 within each well 115 form a single spheroid, as shown in FIG. 1C.

A well insert as described herein may be used to help contain the spheroid in the well 115, i.e., prevent the spheroid from exiting the well 115 during processes such as exchanging cell culture media within the well 115. In some embodiments, the cell culture apparatus 100 may be tilted to remove the cell culture medium when the well insert 150 is positioned in at least a portion of the wells 115. In other embodiments, a pipette may be used to remove the cell culture medium when the well insert 150 is positioned in at least a portion of the wells 115. The use of the well insert 150 in combination with the pipette may reduce the risk of the pipette affecting the spheroid, e.g., the pipette cannot not aspirate the spheroid.

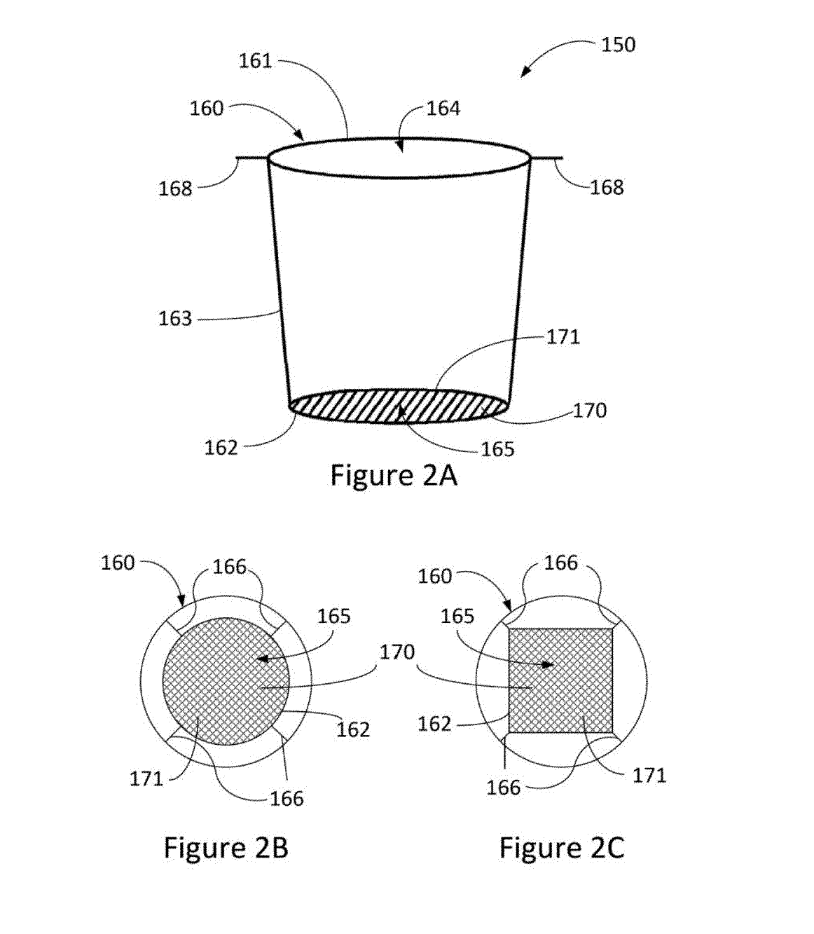

Each of the plurality of wells 115 may include a sphere trap insert, or well insert 150, as shown in FIG. 2A. The well insert 150 may be configured to be positioned in at least a portion of the wells 115. The well insert 150, or portions thereof, may be made of any suitable material. Preferably, materials intended to contact cells or culture media are compatible with the cells and the media. Typically, cell culture components are formed from polymeric material. Examples of suitable polymeric materials include polystyrene, polymethylmethacrylate, polyvinyl chloride, polycarbonate, polysulfone, polystyrene copolymers, fluoropolymers, polyesters, polyamides, polystyrene butadiene copolymers, fully hydrogenated styrenic polymers, polycarbonate PDMS copolymers, and polyolefins such as polyethylene, polypropylene, polymethyl pentene, polypropylene copolymers and cyclic olefin copolymers, and the like

As shown in FIG. 2A, the well insert 150 may include a frame 160. The frame 160 may have a first end 161, a second end 162, and at least one support 163 extending between the first end 161 and the second end 162. As shown, the first end 161 may define an aperture or opening 164. The aperture 164 of the first end 161 may align with a top aperture of the well and allow access to at least a portion of the interior of the well. In some embodiments, the first end 161 may be outside of the well and away from the top aperture of the well. The first end 161 of the frame 160 may be defined by a variety of shapes, e.g., circular, square, rectangular, diamond, hexagonal, etc. In some embodiments, the shape of the first end 161 may match the shape of the top aperture of the well.

The first end 161 of the frame 160 may be configured such that the frame 160 is positioned in at least a portion of the well. For example, the frame 160 may extend from the top aperture of the well down into the well. In some embodiments, the shape of the first end 161 may assist in holding the frame 160 into position proximate the top aperture of the well. As shown in FIG. 2A, the frame 160 may include one or more flanges 168 extending from the first end 161 of the frame 160. The one or more flanges 168 may assist in positioning the frame 160 within at least a portion of the well. For example, the one or more flanges 168 may contact a portion proximate the well to position the frame 160 within at least a portion of the well. In some embodiments, the one or more flanges 168 may contact some other part of the cell culture apparatus to position the frame 160 within at least a portion of the well.

The one or more supports 163 of the frame 160 of the well insert 150 may extend between and be coupled to the first and second ends 161, 162 of the well insert 150, as shown in FIGS. 2A-2C. As shown in FIGS. 2B and 2C, the frame 160 includes four supports 166. The frame 160 may include less than four supports (e.g., one support, two supports, three supports, etc.), four supports, or more than four supports (e.g., five supports, six supports, eight supports, ten supports, etc.). The one or more supports 163 may include, e.g., wire, bars, rods, etc. The one or more supports 163 extending between the first and second ends 161, 162 may define a substantially open frame. In other words, the space between the first and second ends 161, 162 is generally open. In some embodiments, the one or more supports 163 may include a sidewall that extends from and is coupled to the first and second ends 161, 162. In some embodiments, the sidewall may completely surround an edge or perimeter of the second end 162. In other words, the sidewall may enclose an area between the first and second ends 161, 162 and, therefore, define a closed tubular structure of the frame 160. The sidewall may be defined by a variety of different characteristics, e.g., solid, porous, fluid permeable.

The second end 162 of the frame 160 may define an opening 165. The opening 165 of the second end 162 may be closer to a bottom of the well than the first end 161 is to the bottom of the well when the frame 160 is positioned in at least a portion of the well.

The well insert 150 may also include a fluid permeable mesh 170 coupled to the frame 160 and disposed across the opening 165 of the second end 162. The mesh 170 may define pores 171. The pores 171 may define an average pore size of about, e.g., greater than or equal to 5 micrometers, greater than or equal to 10 micrometers, greater than or equal to 20 micrometers, greater than or equal to 35 micrometers, greater than or equal to 50 micrometers, etc. or, less than or equal to 100 micrometers, less than or equal to 90 micrometers, less than or equal to 75 micrometers, less than or equal to 60 micrometers, less than or equal to 45 micrometers, etc. In some embodiments, the pores may define an average pore size of about 40 micrometers. The pores 171 may be defined as being a size to prevent passage of a spheroid through the mesh 170. Also, the pores 171 may be defined as being a size that allows passage of individual cells through the mesh 170. The mesh may be made of a variety of different materials including but not limited to track-etched membrane or a woven or non-woven porous material. The material of the porous membrane may be treated or coated to make it more adherent or more non- adherent to cells. Treatment may be accomplished by any number of methods known in the art which include plasma discharge, corona discharge, gas plasma discharge, ion bombardment, ionizing radiation, and high intensity UV light. Coatings can be introduced by any suitable method known in the art including printing, spraying, condensation, radiant energy, ionization techniques or dipping. The coatings may then provide either covalent or non-covalent attachment sites. Such sites can be used to attach moieties, such as cell culture components (e.g., proteins that facilitate growth or adhesion). Further, the coatings may also be used to enhance the attachment of cells (e.g., polylysine). Alternatively, cell non-adherent coatings as described above can be used to prevent or inhibit cell binding. In some embodiments, the mesh comprises a nylon mesh.

In some embodiments, the mesh may also be disposed between the first and second ends. In such embodiments, in which the mesh or sidewall extends between the first and second ends, the frame may define an interior space or cavity of the frame. In other words, the interior space or cavity of the frame would be defined by the mesh disposed across the second opening and the sidewalls (e.g., mesh, solid, etc.) extending between the first and second ends.

The opening 165 of the second end 162, and thus the mesh 170, may be defined by a variety of shapes, e.g., square, rectangle, circle, hexagon, etc. As shown in FIG. 2B. the opening 165 of the second end 162 may be defined by a circle. As shown in FIG. 2C, the opening 165 of the second end 162 may be defined by a square. The shape of the second end 162 may be described as preventing the spheroid from exiting the well when the well insert 150 is positioned therein. In other words, the shape of the second end 162 of the frame 160 may correspond to the shape of the well such that any gap between the second end 162 of the frame 160 and a side of the well is not large enough to allow a spheroid to pass through. For example, the second end 162 may define a shape of the same size as defined by a side of the well and, thereby, eliminating any gap between the two.

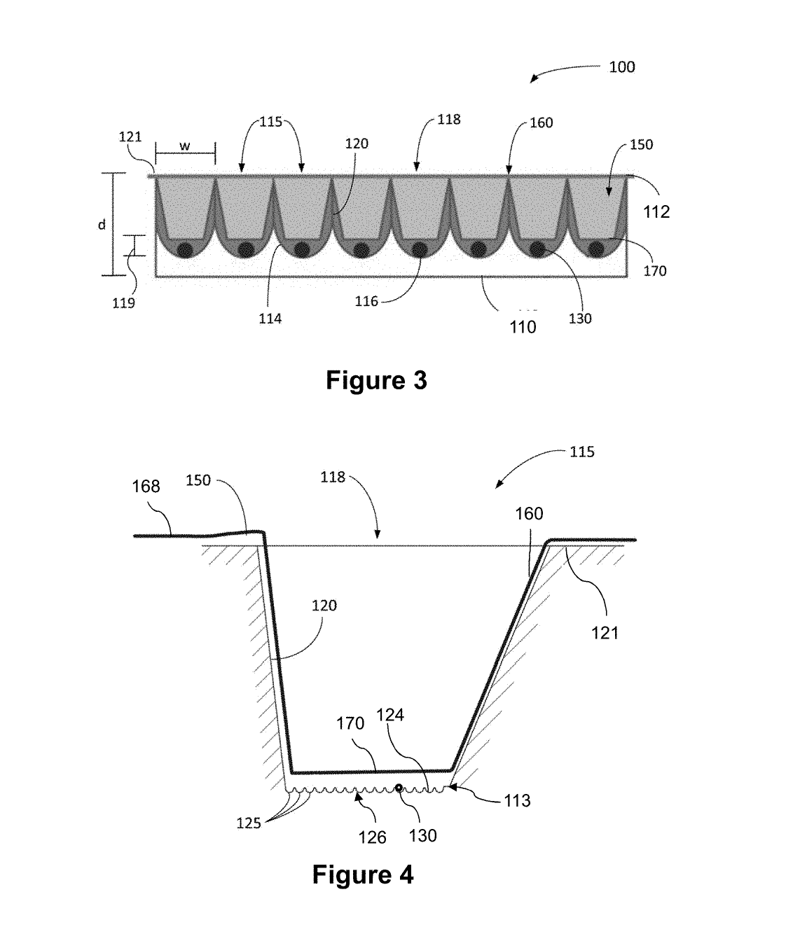

In some embodiments, a cell culture assembly may include one or more wells 115 and one or more well inserts 150, as shown in FIG. 3. As shown, each well 115 includes a corresponding well insert 150. The well insert 150 is configured to be positioned within at least a portion of the well 115. In other words, the well insert 150 may be inserted into at least a portion of the well 115 and the well insert 150 may be removed from at least a portion of the well 115. In some embodiments, the one or more well inserts 150 may be coupled to one another such that the one or more well inserts 150 may be positioned into and out of the one or more wells 115 at the same time. The one or more wells 115 may be coupled through the use of a frame 160. In other embodiments, each of the one or more well inserts 150 may be configured to be individually positioned into and out of a corresponding well 115. In other words, none of the one or more well inserts 150 are attached to one another. In yet other embodiments, the one or more well inserts may be coupled to one another in a variety of combinations based on application. For example, the one or more well inserts may be coupled in groups of about 96, 48, 24, 12, 6, etc.

A structured surface 112 of a cell culture apparatus 100 as described herein may define any suitable number of wells 115 that may have any suitable size or shape. The wells 115 define a volume based on their size and shape. In many embodiments, one or more or all of the wells 115 are symmetrically rotatable around a longitudinal axis. In some embodiments, the longitudinal axes of one or more or all of the wells 115 are parallel with one another. The wells 115 may be uniformly or non-uniformly spaced. Preferably, the wells 115 are uniformly spaced. One or more or all the wells 115 may have the same size and shape or can have different sizes and shapes.

The wells 115 may be defined by substrate 110 that defines a structured surface 112. Each well of the one or more wells 115 defines an interior surface 120, an exterior surface 114 and an upper aperture 118. In some embodiments, the wells 115 may be gas permeable through the substrate 110. The gas permeability of the wells 115 through the substrate 110 to exterior surface 114 will depend in part on the material of the substrate and the thickness of the substrate along the well 115. For example, the gas permeability of the wells may be as described in commonly-assigned U.S. provisional patent application no. 62/072088, which provisional patent application is hereby incorporated herein by reference in its entirety to the extent that it does not conflict with the present disclosure.

The interior surface of the well defines a nadir 116, or a low point, that is opposite the upper aperture 118. Still with reference to FIG. 3, the wells 115 have a depth d defined by the height from nadir 116 to upper aperture 118. The wells 115 also have a diametric dimension w, such as a diameter, width, etc., across the well defined by the upper aperture 118. The wells may have any suitable depth d and diametric dimension w. In some embodiments, the depth d, diametric dimension w and shape of the well, along with the material forming the well, serve to define a volume in which cells can grow.

In some embodiments, the wells 115 described herein have a diametric dimension w in a range from about 200 micrometers to about 500 micrometers. Such diametric dimensions can control the size of a spheroid 130 grown therein such that cells at the interior of the spheroid 130 are maintained in a healthy state. In some embodiments, the wells 115 have a depth d in a range from about 100 micrometers to about 500 micrometers. Of course, other suitable dimensions may also be employed, such as up to 3000 micrometers or greater.

In some embodiments, the inner surface of the wells 115 are non-adherent to cells. The wells 115 may be formed from non-adherent material or may be coated with non-adherent material to form a non-adherent well. In some embodiments, the non-adherent material may be described as an ultra-low-adhesion material. Examples of non-adherent material include perfluorinated polymers, olefins, or like polymers or mixtures thereof. Other examples include agarose, non-ionic hydrogels such as polyacrylamides, or polyethers such as polyethyleneoxide or polyols such as polyvinylalcohol, or like materials or mixtures thereof. The combination of, for example, non-adherent wells, well geometry, and gravity can induce cells cultured in the wells to self-assembly into spheroids 130. Some spheroids 130 can maintain differentiated cell function indicative of a more in vivo like response relative to cells grown in a monolayer.

The interior surface 120 may define a variety of different shapes from the upper aperture 118 to the nadir 116. For example, in some embodiments, one or more wells 115 may be defined by an arcuate surface, such as a hemi-spherical or concave surface, a conical surface having a rounded bottom, and the like surface geometries or a combination thereof. The nadir 116 of the well 115 may ultimately terminate, end, or bottom-out in a spheroid-conducive rounded or curved surface, such as a dimple, a pit, and like concave frusto-conical relief surfaces, or combinations thereof. Other shapes and construction of gas-permeable spheroid-conducive wells are described in commonly-assigned U.S. patent application Ser. No. 14/087,906, which application is hereby incorporated herein by reference in its entirety to the extent that it does not conflict with the present disclosure.

In some embodiments, the interior surface 120 may be flat or come to a point. The interior surface 120 may have any other suitable shape or dimension.

In some embodiments, the mesh 170 may be configured such that individual cells may pass through the mesh 170 when the cells are being seeded into the wells 115. For example, the pores 171 may define an average pore size that is larger than an individual cell. However, after the cells form into spheroids 130, the spheroids 130 may be too large to pass back through the mesh 170. Once the spheroid 130 is positioned between the mesh 170 and the nadir 116 of the interior surface 120, the mesh 170 may also help increase user efficiency by reducing the potential for error. For example, the presence of the mesh 170 prevents a pipette from coming in contact with the spheroid 130. Additionally, the mesh 170 may help diffuse the flow of medium that is being introduced or withdrawn from the well 115. This diffusion may help to prevent eddies from disrupting the spheroid 130.

In some embodiments, the thickness and shape of the substrate around the well is configured to correct for refraction of light passing into the interior surface and out of the exterior surface. For example, the shape and thickness may be as described in commonly-assigned U.S. provisional patent application No. 62/072019, which provisional patent application is hereby incorporated herein by reference in its entirety to the extent that it does not conflict with the present disclosure.

In some embodiments, the well insert 150 may include a fluid permeable mesh 170 and a frame 160. As shown in FIG. 3, the well insert 150 is configured to be at least partially inserted into the well 115 such that the mesh 170 is positioned in the well 170 a distance 119 from the nadir 116. The frame 160 may be coupled to the mesh 170 and extend away from the nadir 116 when the well insert 150 is positioned within the well 115. The frame 160 may be used to help position the well insert 150 into and out of the well 115. In some embodiments, the frame 160 may be configured to position the mesh 170 the distance 119 from the nadir 116. For example, the frame 160 may contact an upper edge 121 of the well to position the mesh 170 the distance 119 from the nadir 116. In another example, the cell culture assembly 100 may include a support that contacts the frame 160 to ensure the mesh 170 is positioned a desired distance 119 from the nadir 116. In other embodiments, the mesh 170 may be in contact with the interior surface 120 of the well 115 and thereby controlling the distance 119 the mesh 170 is from the nadir 116.

As shown in FIG. 4, the well 115 may include at least one sub-well 125 along the interior surface. In some embodiments, a plurality of sub-wells 125 are positioned along the interior surface 120 of the well 115. The sub-wells 125 may have similar characteristics as the wells 115 described above. Alternatively, the cell culture apparatus may include a reservoir and the reservoir may include a plurality of wells as described herein. Also shown in FIG. 4, the well 115 may contain a well insert 150 therein positioned between the interior surface 120 of the well 115 and the upper aperture of the well 115, similar to FIG. 3.

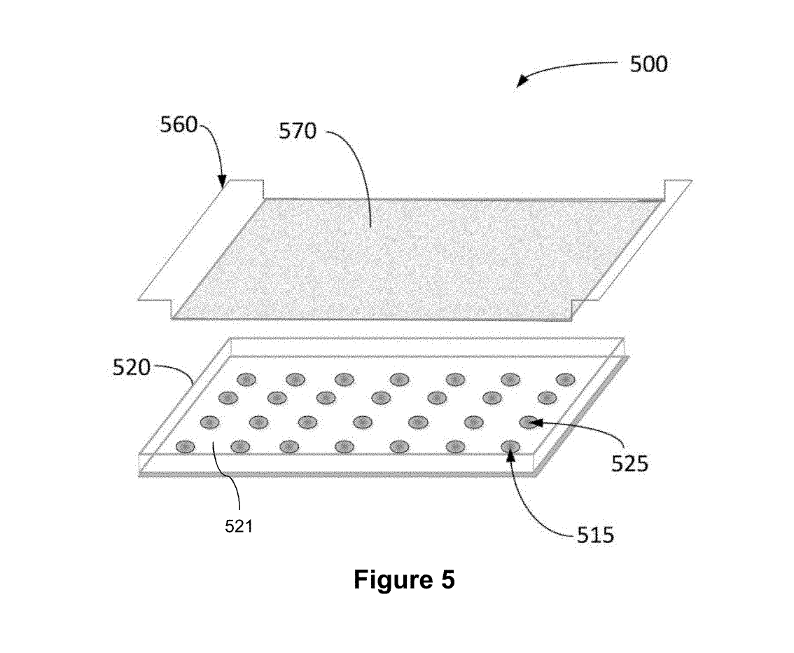

In some embodiments, a cell culture assembly 500 may include a first well 515, a fluid permeable mesh 570 and a frame 560 coupled to the mesh 570, as shown in FIG. 5. The frame 560 may be configured to maintain the mesh 570 in a position over at least a portion of the first well 515. In some embodiments, the mesh 570 is configured to be disposed over an upper edge 520 of the first well 515. In some embodiments, the cell culture assembly 500 may include a second well 525 and the mesh 570 is configured to be positioned over the second well 525.

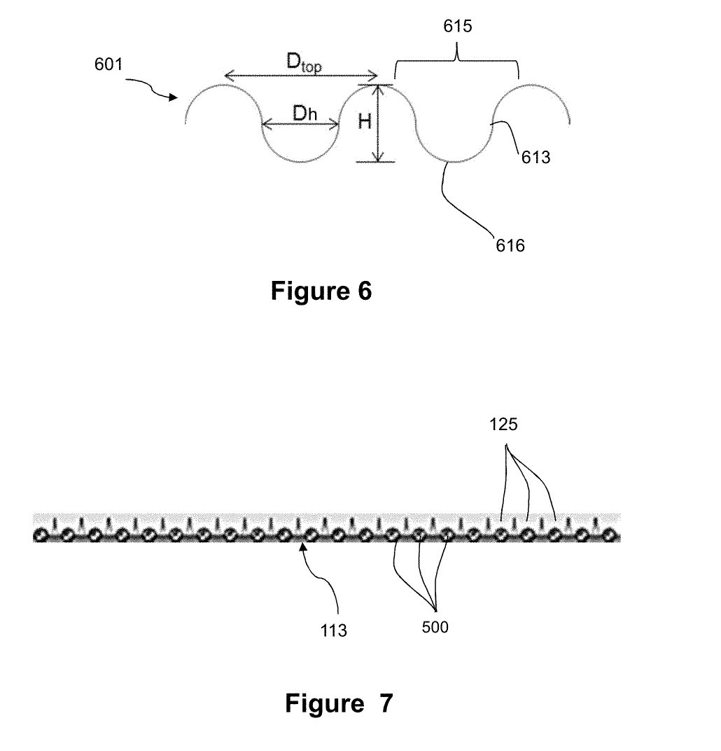

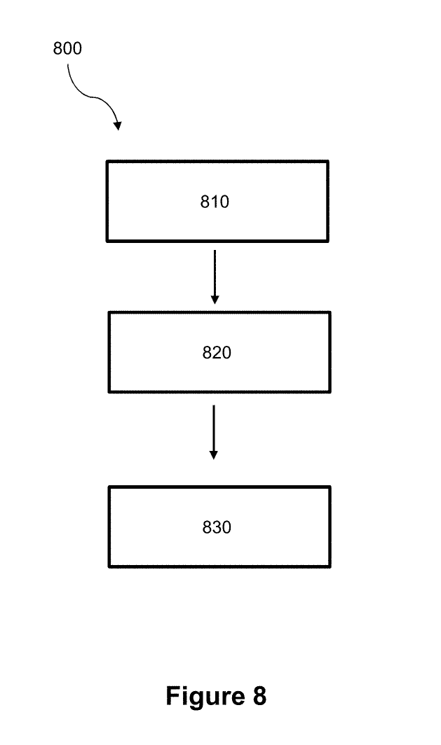

A method 600 for removing culture media from a well of a cell culture apparatus is depicted in FIG. 6. The well defines an interior surface including a nadir. An end of an insert is disposed 610 into the well with the end of the insert defining an opening in fluid communication with a cavity of the insert. The insert includes a fluid permeable mesh disposed across the opening and the end of the insert is disposed within the well such that the mesh is positioned a distance from the nadir. A tip of a fluid removal device is inserted 620 into the cavity of the insert such that the tip is a distance from the nadir at least as far as the distance from the mesh to the nadir. Fluid is removed 630 from the well by withdrawing the fluid via the fluid removal device.

A structured bottom surface as described herein can be formed in any suitable matter. For example, a substrate can be molded to form the structured surface, a substrate film can be embossed to form the structured surface, or the like.

A structured bottom surface as described herein may be assembled into a cell culture apparatus in any suitable manner. For example, the structured bottom surface and one or more other components of the cell culture apparatus may be molded as a single part. In some embodiments, the structured bottom surface or a portion thereof is welded (e.g., thermal welding, ultrasonic welding, or the like), adhered or thermoformed to one or more other components of the cell culture apparatus.

All scientific and technical terms used herein have meanings commonly used in the art unless otherwise specified. The definitions provided herein are to facilitate understanding of certain terms used frequently herein and are not meant to limit the scope of the present disclosure.

As used herein, singular forms “a,” “an” and “the” include plural referents unless the context clearly dictates otherwise. Thus, for example, reference to a “structured bottom surface” includes examples having two or more such “structured bottom surfaces” unless the context clearly indicates otherwise.

As used in this specification and the appended claims, the term “or” is generally employed in its sense including “and/or” unless the content clearly dictates otherwise. The term “and/or” means one or all of the listed elements or a combination of any two or more of the listed elements.

As used herein, “have”, “has”, “having”, “include”, “includes”, “including”, “comprise”, “comprises”, “comprising” or the like are used in their open ended inclusive sense, and generally mean “include, but not limited to”, “includes, but not limited to”, or “including, but not limited to”.

“Optional” or “optionally” means that the subsequently described event, circumstance, or component, can or cannot occur, and that the description includes instances where the event, circumstance, or component, occurs and instances where it does not.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the inventive technology.

Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Also herein, the recitations of numerical ranges by endpoints include all numbers subsumed within that range (e.g., 1 to 5 includes 1, 1.5, 2, 2.75, 3, 3.80, 4, 5, etc.). Where a range of values is “greater than”, “less than”, etc. a particular value, that value is included within the range.

Any direction referred to herein, such as “top,” “bottom,” “left,” “right,” “upper,” “lower,” “above,” below,” and other directions and orientations are described herein for clarity in reference to the figures and are not to be limiting of an actual device or system or use of the device or system. Many of the devices, articles or systems described herein may be used in a number of directions and orientations. Directional descriptors used herein with regard to cell culture apparatuses often refer to directions when the apparatus is oriented for purposes of culturing cells in the apparatus.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. Any recited single or multiple feature or aspect in any one claim can be combined or permuted with any other recited feature or aspect in any other claim or claims.

It is also noted that recitations herein refer to a component being “configured” or “adapted to” function in a particular way. In this respect, such a component is “configured” or “adapted to” embody a particular property, or function in a particular manner, where such recitations are structural recitations as opposed to recitations of intended use. More specifically, the references herein to the manner in which a component is “configured” or “adapted to” denotes an existing physical condition of the component and, as such, is to be taken as a definite recitation of the structural characteristics of the component.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to an insert comprising a frame and a mesh include embodiments where an insert consists of a frame and a mesh and embodiments where an insert consists essentially of a frame and an insert.

It will be apparent to those skilled in the art that various modifications and variations can be made to the present inventive technology without departing from the spirit and scope of the disclosure. Since modifications, combinations, sub-combinations and variations of the disclosed embodiments incorporating the spirit and substance of the inventive technology may occur to persons skilled in the art, the inventive technology should be construed to include everything within the scope of the appended claims and their equivalents.

What is claimed is:

1. A cell culture assembly comprising:
a cell culture container comprising at least one well having an opening and a nadir; an insert structured to fit inside the at least one well of the cell culture container, said
insert comprising a first open end, a second end having an opening, wherein said first open end and said second end define a cavity therebetween,
wherein the second end comprises a fluid permeable mesh;
wherein the cell culture container comprises a cell culture surface and wherein said cell culture surface comprises a substrate having an array of microwells.

2. The cell culture assembly of claim 1 wherein the mesh comprises pores having an average pore size in a range from 10 micrometers to 200 micrometers.

3. A cell culture assembly according to claim 1, wherein the well is configured such that cells cultured in the well form spheroid in the array of microwells.

4. A cell culture assembly according to claim 3, wherein the pores of the mesh are of a size that allows passage of individual cells through the mesh and prevents passage of spheroids formed in the array of microwells through the mesh.

5. The cell culture assembly of claim 1, wherein well insert further comprises a side wall, or a plurality of supports, extending from the first end to the second end.

6. The cell culture assembly of claim 5, wherein at least a portion of said side wall comprises said mesh.

7. The cell culture assembly of claim 1, wherein the well insert further comprises one or more flanges extending from the first open end.

8. The cell culture assembly of claim 1, wherein the well further defines an upper edge, wherein the first open end of the insert contacts the upper edge of the well to position the mesh a distance from the nadir.

9. The cell culture assembly of claim 8, further comprising a support, wherein the insert contacts the support to position the mesh a distance from the nadir.

10. The cell culture assembly of claim 1, wherein the mesh comprises an average pore size of less than or equal to 40 micrometers.

11. The cell culture assembly of claim 1, wherein said well comprises a conical shape from the aperture to the nadir.

12. The cell culture assembly of claim 1, wherein at least a portion of the interior surface of the insert is treated or coated to prevent cell adhesion to the interior surface of the insert.

13. A method of culturing spheroid cells in a cell culture assembly of claim 1 comprising:
positioning an insert in a well;
introducing cells and media into the cavity of the insert,
allowing cells to pass through the mesh of the insert and form spheroids in the space between the nadir of the well and the mesh of the insert.

14. A method of removing media from a cell culture assembly of claim 1 comprising:
positioning an insert in a well;
introducing cells and media into the cavity of the insert,
allowing cells to pass through the mesh of the insert and form spheroids in the space between the nadir of the well and the mesh of the insert
removing the media from the well above the insert.

15. A cell culture assembly comprising:
a well comprising an aperture, sidewalls and a nadir;
wherein the nadir comprises an array of microwells;
an insert comprising:
an aperture, a frame and a fluid permeable mesh comprising pores,
wherein the pores comprise an average pore size in a range from 10 micrometers to 100 micrometers, and wherein the insert is configured to be positioned within at least a portion of the well at a distance from the nadir.

16. The cell culture assembly of claim 15, wherein the pores of the mesh are configured to prevent spheroids from passing through the mesh.

17. The cell culture assembly according to claim 15, wherein the well further defines an upper edge, wherein the frame contacts the upper edge to position the mesh a distance from the nadir.

18. The cell culture assembly according to claim 15, further comprising a support, wherein the frame contacts the support to position the mesh a distance from the nadir.

19. The cell culture assembly according to claim 15, wherein pores comprise an average pore size of less than or equal to 40 micrometers.

20. The cell culture assembly according to claim 15, wherein interior surface of the well comprises a conical shape from the aperture to the nadir.

21. The cell culture assembly according to claim 15, wherein the mesh comprises a circular shape.

22. The cell culture assembly according to claim 15, wherein the mesh defines a square shape.

23. The cell culture assembly according to claim 15, further comprising a plurality of wells and wherein the insert comprises a plurality of inserts, wherein each of the inserts is configured to be positioned in at least a portion of a different one of the wells of the plurality of wells.

24. The cell culture assembly according to claim 15, wherein the interior surface of the well is coated with an ultra-low-adhesion material.

25. The cell culture assembly according to claim 15, wherein at least a portion of the frame comprises mesh.

26. The cell culture assembly according to claim 15, wherein the frame comprises at least one support wire coupled to the fluid permeable mesh.

27. The cell culture assembly according to claim 15, wherein the well comprises at least one sub-well along the interior surface.

28. A method of culturing spheroid cells in a cell culture assembly according to claim 15 comprising:
positioning an insert in a well;
introducing cells and media into the cavity of the insert,
allowing cells to pass through the mesh of the insert and form spheroids in the space between the nadir of the well and the mesh of the insert.

29. A method of removing media from a cell culture assembly according to claim 15 comprising:
positioning an insert in a well;
introducing cells and media into the cavity of the insert,
allowing cells to pass through the mesh of the insert and form spheroids in the space between the nadir of the well and the mesh of the insert
removing the media from the well above the insert.
